(19) United States
(12) Patent Application Publication
(10) Pub. No.: US 2015/0065454 A1
(43) Pub. Date: Mar. 5, 2015

(54) Solution aqueuse homogene de chitosane injectable

(71) Applicants: Cytosial Biomedic, Nimes (FR); Universite Jean Monnet, Saint Etienne (FR); Institut National des Sciences Appliquees de Lyon, Villeurabanne (FR); Universite Claude Bernard Lyon 1, Villeurbanne (FR); Centre National de la Recherche Scientifique (CNRS, Paris (FR)

(72) Inventors: Florence Dupasquier, Paray le Monial (FR); Laurent David, Lyon (FR); Thierry Delair, Echalas (FR)

(21) Appl. No.: 14/361,400

(22) PCT Filed: Nov. 30, 2012
(86) PCT No.: PCT/EP2012/074059

§ 371 Date: Nov. 18, 2014

(30) Foreign Application Priority Data
Nov. 30, 2011 (FR) 1160988

(51) Int. Cl. A61L 27/20; A61Q 19/08; A61K 8/73; A61L 27/58; A61L 27/54

(52) U.S. Cl. CPC A61L 27/20; A61L 27/58; A61L 27/54; A61K 8/736; A61Q 19/08; A61L 2430/34; A61L 2400/06; A61K 2800/91
USPC 514/55

(57) ABSTRACT

The present invention relates to a homogeneous aqueous solution of injectable chitosan containing a chitosan having a degree of acetylation lower than 20%, said solution containing between 0.1 and 3.5% by weight of chitosan, said solution presenting a pH lower than 6.2, and said aqueous solution being capable of forming crystalline particles of chitosan after injection. The present invention also relates to compounds containing such a homogeneous aqueous solution of chitosan. The invention also relates to such compounds for their use as dermatological or cosmetic compounds, or for their use as a medical device, advantageously as a bioresorbable implant.

The present invention relates to the field of fillers or biomaterials, injectable in humans or animals. In particular, the present invention relates to a homogeneous aqueous solution of injectable chitosan capable of forming crystalline particles of chitosan after injection. The present invention also relates to compositions containing such a homogeneous aqueous solution of chitosan. The invention also has as an object such compositions for use as a dermatological or cosmetic composition, or as a medical device, advantageously as a bioresorbable implant.

Various fillers injectable in particular in humans are already known.

Collagen has long been the first choice as filler for the face, in particular for filling lines and wrinkles or for filling around the lips. However, since being introduced on the market, hyaluronic acids have become the most used. Indeed, in addition to the fact that collagen's biodegradability is considered too fast, there are safety issues related to its animal origin (from cows or pigs).

The direct injection of hyaluronic acid has two advantages: an immediate mechanical filling effect and an absence of inflammatory phenomena, by virtue of its biocompatibility. However, this biocompatibility is associated with rapid biodegradation, making the product unsatisfactory, even if the lifespan of the injected product were to be extended by means of the use of crosslinked hyaluronic acid.

Nevertheless, the products most used today in cosmetic medicine and surgery are resorbable products whose lifespan is generally less than 12 months.

Also found on the market are fillers described as “permanent” in the sense that their bioresorption can require several years. These products contain, among others, synthetic or biosynthetic polymers such as acrylic derivatives and polyacrylamides, which induce significant fibrous encapsulation, the source of the filling's longevity. However, the persistence of the injected product in tissues presents a risk of long-term complications or delayed inflammatory phenomena, for example the formation of inflammatory granulomas, cysts, etc., several months or even several years after their injection.

Other products constitute today an advantageous alternative, namely polylactic acid (PLA), a polymer whose biodegradation is slower than that of other natural polymers such as collagen or hyaluronic acid. Indeed, it is estimated that filling persists up to two years after injection. These products are marketed in particular under the name New-Fill (or Sculptra). The principal defect of this technology is that the filling effect is visible only after a wait of eight weeks, which does not provide complete satisfaction to the patient.

Furthermore, the fibrosis observed during the use of non-degradable products seemed to be of great advantage in terms of the long-term esthetic effect, which thus lead to the development of so-called “semi-permanent” fillers which, by their heterogeneous “particle-vector” composition have a pro-fibrotic effect while remaining biodegradable. Mention may be made, for example, of the product Atlean which offers a dispersion of tricalcium phosphate (TCP) particles in hyaluronic acid and the product Radiesse which offers a dispersion of calcium hydroxyapatite particles in a carboxymethyl cellulose gel. In all cases, the gel vector provides the esthetic effect of immediate filling, while the particles gradually generate fibrosis which guarantees the long-term effect. The advantage of these products, besides this dual action mechanism (mechanical and tissue inductor), is that they are in the end completely reabsorbed.

In a particularly advantageous manner, chitosan, due to its unique chemical structure, behaves with respect to the organism as a “decoy” of biological media (A. Montembault, K. Tahiri, C. Korwin-Zmijowska, X, Chevalier, M. Corvol, A. Domard, Biochimie, 88 (2006), 551-64): on the one hand, it is sufficiently “recognized” not to induce a dangerous inflammatory reaction, and on the other hand it is sufficiently “unrecognized” not to be degraded too quickly.

The molecule indeed consists of a sequence of N-acetyl-D-glucosamine and D-glucosamine fragments, the first being a constituent of molecules of the extracellular matrix (this residue is found in hyaluronic acid, for example), and the second being completely absent therefrom; the chitosan is thus more difficult to degrade from a biological point of view.

The concept of the use of a “decoy” of biological media is completely novel in the field of injectables, in particular in cosmetic medicine, and no filler containing chitosan has been marketed to date.

Furthermore, chitosan is known in the literature to stimulate certain immune cells, such as macrophages, which produce in its presence an increased quantity of growth factors. These growth factors are biological mediators which promote the production of the extracellular matrix and the proliferation of fibroblasts, cells that produce collagen fibers. Thus, chitosan promotes the synthesis of fibrous tissue, which enables long-term “biological” filling without undesirable side effects, in particular filling of defects of the skin or cavities of the human body or face, such as wrinkles.

The object of the present invention is thus a homogeneous aqueous solution of injectable chitosan containing a chitosan having a degree of acetylation lower than 20%, advantageously lower than 10%, said aqueous solution containing between 0.1 and 3.5%, advantageously between 1 and 2.5%, by weight of chitosan, said solution having a pH lower than 6.2, advantageously between 5 and 6.2, and said aqueous solution being capable of forming crystalline particles of chitosan after injection.

Chitosan is an amino-polysaccharide generally obtained by N-deacetylation of chitin, a polysaccharide as widespread in the biomass as cellulose. Chitin is in particular present in the cuticles of arthropods, the endoskeleton of cephalopods, the cell walls and extracellular matrix of fungi, yeasts and algae.

Advantageously according to the present invention, the chitosan is a natural substance that comes from an animal source, for example shellfish such as crabs, shrimps or squids, or from a plant source, such as fungi or algae. Chitosan and chitin are linear copolymers of 2-acetamido-2-deoxy-D-glucan and 2-amino-2-deoxy-D-glucan, respectively. They are more commonly described as N-acetyl-D-glucosamine (GlcNAc) and D-glucosamine (GlcN) units linked by β-(1→4) glycosidic bonds. Chitin and chitosan are differentiated by the molar fraction (expressed in %) of GlcNAc units present in the copolymer, also called degree of acetylation (DA).

The chemical structures of chitosan and chitin are represented schematically below as a function of degree of acetylation (DA):

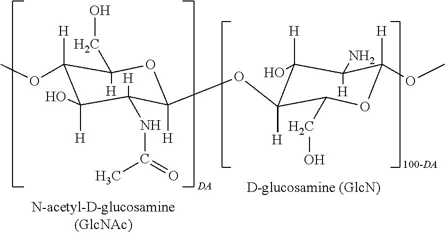

Degree of acetylation (DA):

$$DA ( \% ) = \frac{nGlcNAc}{nGlcNAc + nGlcN} \times 100$$

with nGlcNAc=number of acetylated motifs and nGlcN=number of deacetylated motifs.

Advantageously according to the present invention, the chitosan has a degree of acetylation (DA) lower than 20%, even more advantageously lower than or equal to 15%, for example lower than 10%. Typically, the chitosan according to the invention has a degree of acetylation (DA) between 0.5 and 20%, typically between 1 and 15%, for example between 2 and 10%.

Typically, the chitosan has a mean molecular weight (determined as described in “Physico-chemical studies of the gelation of chitosan in a hydroalcoholic medium” A. Montembault, C. Viton, A. Domard, Biomaterials, 26(8), 933-943, 2005) between 100,000 and 1,000,000 g/mol, advantageously between 250,000 and 1,000,000 g/mol, for example between 250,000 and 500,000 g/mol, for example between 250,000 and 400,000 g/mol.

According to a particular characteristic, another chitosan of lower mean molecular weight, advantageously lower than 20,000 g/mol, can be added to the chitosan as previously defined.

In a particularly advantageous manner, the pH of the aqueous solution according to the present invention is lower than 6.2, and is typically between 5 and 6.2. In the context of the invention, the chitosan is soluble in an aqueous solution, such as water, in an acid environment in the pH ranges mentioned previously, advantageously by protonation of the chitosan's amine groups. Advantageously, the aqueous solution according to the invention is stable.

By “homogeneous” solution of chitosan is meant, in the context of the present invention, that the entire chitosan polymer is solubilized, with the liquid phase containing no suspended solids. The solution according to the invention is thus not gelled. The solution according to the invention is thus typically transparent.

According to a particular characteristic of the present invention, the homogeneous aqueous solution of chitosan contains between 0.1 and 3.5%, advantageously between 0.5 and 3.5%, in particular between 1 and 2.5%, by weight of chitosan, in relation to the total weight of the aqueous solution.

In a particularly advantageous manner, the aqueous solution according to the invention is injectable in the human or animal body, typically intradermally or subcutaneously. The solution can be packaged in a syringe such as a sterile syringe.

Advantageously, the aqueous solution has a viscosity suitable to good syringeability (satisfactory flow through a needle in a syringe) and ease of injection.

In a particular embodiment, the aqueous solution according to the invention is sterilized before injection, for example by autoclave.

After sterilization, the chitosan typically has a mean molecular weight between 80,000 and 400,000 g/mol, and advantageously between 120,000 and 300,000 g/mol.

In a particularly advantageous manner, the aqueous solution according to the present invention before injection does not contain a chitosan having a degree of acetylation higher than 20%. Thus, the chitosan according to the invention is not mixed with a chitosan having a degree of acetylation between 30 and 60%, such as that described in patent applications WO 2008/072230 and WO 2009/150651.

In a particular embodiment according to the present invention, the aqueous solution contains several chitosans, but with a single degree of acetylation (DA), said degree of acetylation being lower than 20%, advantageously lower than 10%.

In another particular embodiment of the present invention, the aqueous solution contains a chitosan as previously defined in mixture with another chitosan, such as a chitosan oligosaccharide, also called a chito-oligosaccharide, having a degree of acetylation lower than 20%, advantageously lower than 10%, in an even more advantageous manner having a degree of acetylation identical to the chitosan as previously defined, and typically having a very low mean molecular weight, for example lower than 20,000 g/mol, advantageously lower than 17,000 g/mol, in order to increase the crystallinity of the product once injected.

In another particular embodiment of the present invention, the aqueous solution contains as polymer a single chitosan having a degree of acetylation as previously defined, having advantageously a mean molecular weight as previously defined, advantageously at a concentration of between 0.1 and 3.5%, advantageously between 0.5 and 3.5%, in particular between 1 and 2.5%, by weight of chitosan, in relation to the total weight of the aqueous solution.

In a particular embodiment, the aqueous solution according to the invention can be partially crosslinked by ionic interactions induced, for example, by the addition of sulfate, citrate, metal anions or anionic molecules, in particular by the formation of polyelectrolyte complexes with polysaccharides having a carboxylic group COO⁻ (alginates, pectin, xanthan, hyaluronic acid), with polysaccharides having a sulfate group, or with polylactic acid (PLA), or by interaction with proteins (collagen), nucleic acids (DNA, RNA, siRNA, mRNA, etc.) or oxidized polysaccharides.

In another particular embodiment, the aqueous solution according to the invention is partially crosslinked using covalent crosslinking agents (genipin, for example), to the exclusion of agents known for their toxicity, such as agents of the group of bi- or poly-functional epoxies or esters, divinyl sulfone, carbodiimides, and dialdehydes.

Advantageously, the crosslinking agent, whether of the ionic or covalent type, is introduced in such a way that the crosslinking rate is sufficiently low not to impair the ability of the aqueous solution to form crystalline particles of chitosan after injection.

In a particular embodiment, the aqueous solution according to the invention is composed of the combination of an aqueous solution of non-crosslinked chitosan with an aqueous solution of crosslinked chitosan.

According to a particular characteristic, the aqueous solution according to the invention can be prepared by the following steps:
dissolution of the chitosan in water by the addition of an organic acid such as a weak acid, said weak acid being advantageously selected from the group comprised of acetic acid, glycolic acid, lactic acid, glutamic acid, and mixtures thereof,
and, optionally, readjustment of the pH in order to obtain an aqueous solution having a pH between 5 and 6.2, typically between 5 and 5.5.

Before dissolution, the chitosan is typically in powder form. After dissolution, the chitosan is in protonated form. It is a cationic polyelectrolyte whose counterion results from the acid used for the dissolution. For example, if acetic acid is added to water to dissolve the chitosan, the chitosan will be found in the form of chitosan acetate, i.e., a protonated form NH₃⁺ of the amine functional groups in electrostatic interaction with the acetate ions.

It is very important to control the pH of the solutions to prevent acid necrosis of tissues after injection and also to protect the solutions from hydrolysis and chitosan degradation if sterilization is used (by autoclave at 121° C. for 15 minutes, for example).

The pH is readjusted if necessary with a compound such as sodium bicarbonate or phosphate-buffered saline (PBS), typically in reduced quantities. The pH value is advantageously monitored with a pH-meter during the increase in pH in order to remain at a pH lower than 6.2 and to avoid gelling of the solution.

In a particular embodiment according to the invention, the chitosan is dissolved in water using a strong acid of the hydrochloric acid type. In this case, the pH is readjusted with a compound of the sodium or ammonium bicarbonate type or PBS, for example, and/or a base of the NaOH or KOH type, for example (always controlling the pH so that it remains lower than 6.2).

In a particular embodiment according to the invention, during the dissolution step, the acid is added in an amount necessary to dissolve the chitosan. Use can thus be made of an excess of acid for certain chitosans, for example chitosans that are difficult to solubilize with the strictly necessary amount of acid, and then the chitosan is reprecipitated, using ammonia for example. After a series of washes intended to eliminate excess ammonia and salts, the chitosan can then be lyophilized to recover the dry matter. The latter will then be easier to solubilize.

In another particular embodiment according to the invention, during the dissolution step, the acid is added in an amount strictly necessary to dissolve the chitosan, such as the stoichiometric amount strictly necessary to the protonation of NH₂ sites.

Typically, the number of sites to protonate is calculated as follows:

$$Mmonomer = 203 \times DA + 161 \times ( 1 - DA )$$

$${N}_{NH 2} = \frac{m \times ( 1 - DA ) \times ( 1 - \% water )}{Mmonomer}$$

with m=weight of raw material introduced, % water=water content of the raw material, DA=degree of acetylation.

After injection, in particular in the dermis or subcutaneously, the homogeneous aqueous solution according to the present invention will advantageously form a semicrystalline system, in particular due to the pH change related to the influence of the buffered media of the organism.

By “semicrystalline system” is typically meant a system composed of a crystalline phase and a non-crystalline (amorphous) phase.

Typically, the chitosan crystals obtained correspond to the hydrated allomorph of chitosan.

In a particularly advantageous manner according to the present invention, the aqueous solution has good biocompatibility and is bioresorbable. In particular, the product according to the invention has a longer bioresorption period than products containing hyaluronic acid of the crosslinked hyaluronic acid type, for an extended effect, such as an extended filling effect.

By “bioresorbable” or “bioresorption” is meant biodegradation that leads to the total or essentially total degradation of the product injected.

According to a particular characteristic of the present invention, the chitosan solution is fluid before injection and has a long resorption time once injected, typically of a few weeks to several months, for example of about 3 or 4 weeks up to 12 to 18 months.

The product or biomaterial composed of or containing the aqueous solution according to the invention benefits from the bacteriostatic and fungistatic nature of chitosan, well-known in the world of agribusiness and wound repair dressings. These properties facilitate the preservation of the product and help limit the risks of infection related to injection or to delayed inflammatory phenomena for other products as recalled above. In terms of the natural molecules used to date to fill wrinkles (collagen, hyaluronic acid), chitosan is the only one to have such properties.

Furthermore, the product or biomaterial composed of or containing the aqueous solution according to the invention provides effective biological filling that is advantageously immediate: indeed chitosan, by promoting collagen synthesis, enables the filling of skin defects, such as wrinkles, by stimulating natural mechanisms.

The present invention also has as an object the use of a homogeneous aqueous solution of injectable chitosan containing a chitosan having a degree of acetylation lower than 20%, advantageously lower than 10%, said solution containing between 0.1 and 3.5%, advantageously between 1 and 2.5%, by weight of chitosan, said solution having a pH lower than 6.2, advantageously between 5 and 6.2, to form crystalline particles of chitosan after injection.

Advantageously, the chitosan is as previously defined.

In particular, the chitosan has a mean molecular weight between 100,000 and 1,000,000 g/mol, for example between 250,000 and 1,000,000 g/mol, typically between 250,000 and 500,000 g/mol.

Typically, said aqueous solution does not contain a chitosan having a degree of acetylation higher than 20%.

In an advantageous manner, said aqueous solution can be prepared according to the steps of the process mentioned previously.

The present invention also has as an object a composition comprising an aqueous solution according to the invention, and optionally an acceptable compound or excipient, such as a compound or excipient to promote the crystallinity of the solution after injection, typically short-chain chitosans of degree of acetylation lower than 20% and mean molecular weight lower than 20,000 g/mol, or a chito-oligosaccharide of degree of polymerization between 3 and 30.

In a particular embodiment, the composition according to the invention includes a salt such as sodium chloride, or any other acceptable excipient advantageously to adjust the osmolarity of the composition. The addition of a salt such as sodium chloride can be advantageous for obtaining an isotonic solution.

According to a particular characteristic of the present invention, the composition can further include at least a compound having a recognized therapeutic activity. Examples include an analgesic compound, a local anesthetic compound such as lidocaine, mepivacaine, bupivacaine or ropivacaine, an angiogenic compound, a vaccine, a hormone, or an active compound of the growth factor or bioactive oligosaccharide type, for example a hyaluronic acid oligosaccharide or chitosan oligosaccharide of degree of polymerization lower than 20, or a nucleic acid or a protein.

Advantageously according to the present invention, the composition is formulated to be administered or is used by intradermal or subcutaneous injection.

The present invention also has as an object such a composition or aqueous solution according to the invention for use as a dermatological or cosmetic composition, or as a medical device, advantageously as a bioresorbable implant.

The present invention also has as an object a cosmetic use, or a cosmetic or esthetic method for treating the human body or face, comprising the injection of a composition or an aqueous solution according to the invention.

In a particular embodiment, the composition or aqueous solution according to the present invention is intended to be used in the repair or the reconstruction of tissues of the skin of the face or body.

In particular, the composition or aqueous solution according to the present invention can be used to fill cavities of the body or face, such as lines or wrinkles, to create or increase the volume of the human face or body, or to cicatrize the skin.

According to other particular embodiments, the composition or aqueous solution according to the present invention can be used in:
surgery, in particular in the repair of organs, or in cosmetic medicine or surgery,
urology, in particular for the treatment of urinary incontinence,
infectious diseases, in particular as a vector fluid for vaccines,
ophthalmology, in particular for corneal cicatrization,
odontology, in particular for placing a dental implant or for osseous repair,
or angiology.

The composition or aqueous solution according to the present invention can also be used in rheumatology.

Advantageously, the composition or aqueous solution according to the present invention can also be used as a vector for an active ingredient, in particular for a therapeutic active ingredient, such as a vaccine or a hormone of the insulin or estrogen type, and more generally for all active ingredients whose delivery or controlled and/or extended release has an advantage.

Lastly, the present invention relates to the cosmetic use of an aqueous solution or composition according to the invention to treat or prevent skin aging.

The following examples are intended to illustrate the invention without in any way limiting its scope.

### Example 1: In Vitro Study of the Crystallization of Chitosan Solutions According to Invention in PBS

In the context of this in vitro experiment, phosphate-buffered saline (PBS) was selected to simulate the physiological medium. PBS is an isotonic buffer medium of pH 7.2-7.4, commonly used in biology.

The solutions tested are chitosan solutions of various degrees of acetylation (DAs), of concentration C=3% by weight of chitosan in relation to the weight of the solution, and of pH between 5 and 5.5.

The chitosan used is a chitosan resulting from squid chitin (Mahtani Chitosan Veraval, India) with a mean molecular weight of 400,000 g/mol, evaluated by means of a protocol described in “Physico-chemical studies of the gelation of chitosan in a hydroalcoholic medium” A. Montembault, C. Viton, A. Domard, Biomaterials, 26(8), 933-943, 2005. The solutions were prepared using acetic acid.

The various chitosan solutions tested have a DA of 2%, 3.5%, 15%, 40% and 55%, with all these solutions having a chitosan content of 3% by weight.

Were also tested a mixture of chitosan solutions of DA 15% and 40%, with respective chitosan concentrations of 0.5% and 2% by weight, as well as a mixture of chitosan solutions of DA 15% and 55%, with respective chitosan concentrations of 0.5% and 2% by weight, in order to compare the products according to the invention and the products as described in the patent applications of the prior art WO 2008/072230 and WO 2009/150651.

The various DAs are obtained by reacetylation of a squid chitosan (Mahtani Chitosan Veraval, India) of DA 3.5%, Mw approximately 400,000 g/mol, purified by filtration of a chitosan acetate solution at a concentration of 0.5% by weight of polymer through a 0.45 μm filter. The solution is then lyophilized.

In a reactor, and with mechanical stirring (approximately 50 rpm), the chitosan lyophilizate is dissolved in deionized water using the stoichiometric amount of acetic acid necessary to the protonation of NH₂ sites. Various concentrations (0.5 w % to 3 w %) were studied.

The pH of each solution was controlled, and is in all cases between 5 and 5.5 (as a function of the chitosan concentration).

0.30 ml of each chitosan solution was added to 30 ml of PBS and left in this medium for 24 and 72 hours. The samples were then removed from the PBS, placed in capillary tubes filled with pure sterile water and analyzed by synchrotron x-ray diffraction using a 16 keV (λ=0.7749 Å) monochromatic beam on a D2AM beamline (ESRF, Grenoble). The intensity diffracted by the samples is given as a function of the scattering vector q=(4π sinθ)/λ, where 2θ is the angle of diffraction (between the incident beam and the diffracted beam) and after subtraction of the intensity diffracted by the capillary tube filled with water alone, so as to best subtract the contribution of the water and the container to the diffraction.

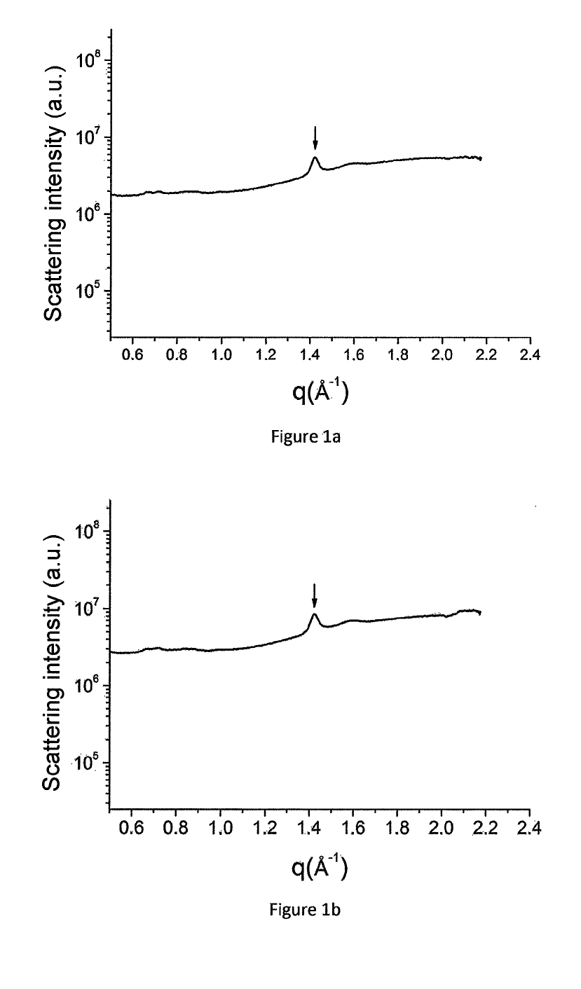

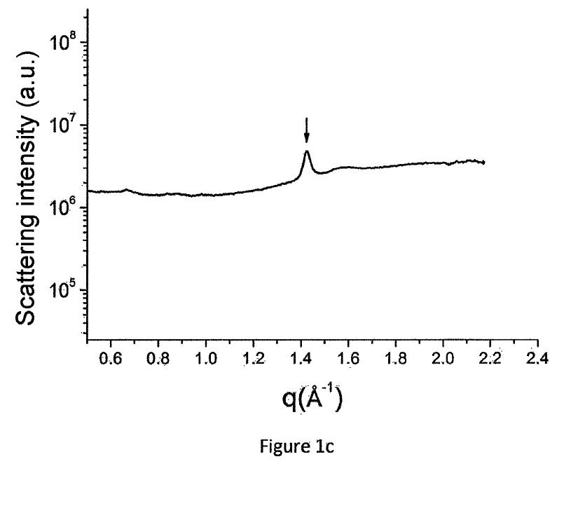

The results of this in vitro study are presented in FIGS. 1 to 3.

FIGS. 1a, 1b and 1c present the results of chitosan solutions according to the invention, with a low DA (lower than or equal to 15%).

FIG. 1a presents the intensity diffracted by a chitosan solution of DA 2%, C=3 w %, after 24 hours in PBS.

FIG. 1b presents the intensity diffracted by a chitosan solution of DA 3.5%, C=3 w %, after 24 hours in PBS.

FIG. 1c presents the intensity diffracted by a chitosan solution of DA 15%, C=3 w %, after 24 hours in PBS.

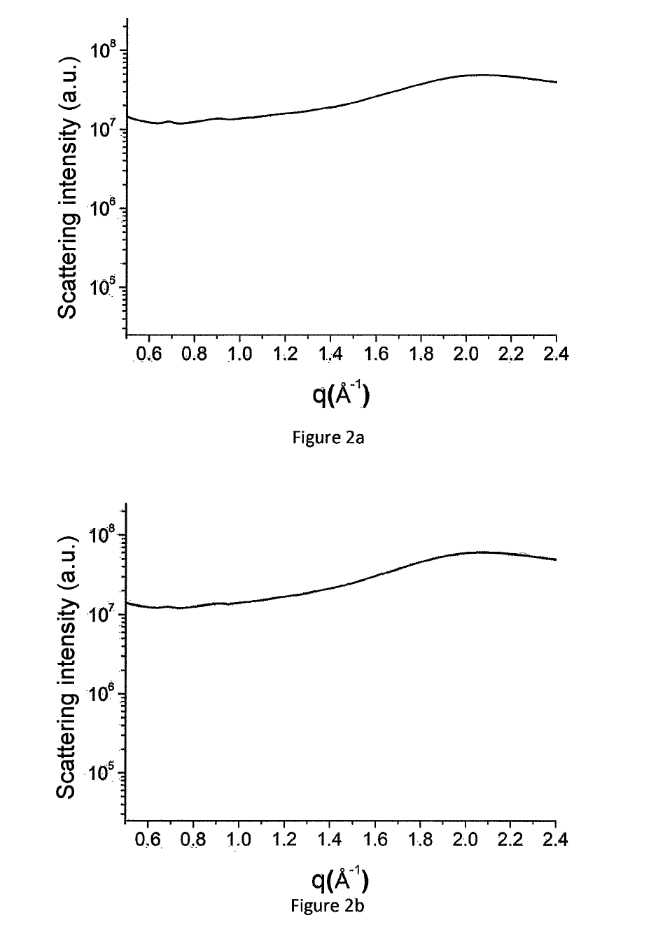

FIGS. 2a and 2b present the results of chitosan solutions which are not within the scope of the present invention, with a high DA (from 40 to 55%), which are used as comparative products with the products according to the present invention.

FIG. 2a presents the intensity diffracted by a chitosan solution of DA 40%, C=3 w %, after 24 hours in PBS.

FIG. 2b presents the intensity diffracted by a chitosan solution of DA 55%, C=3 w %, after 24 hours in PBS.

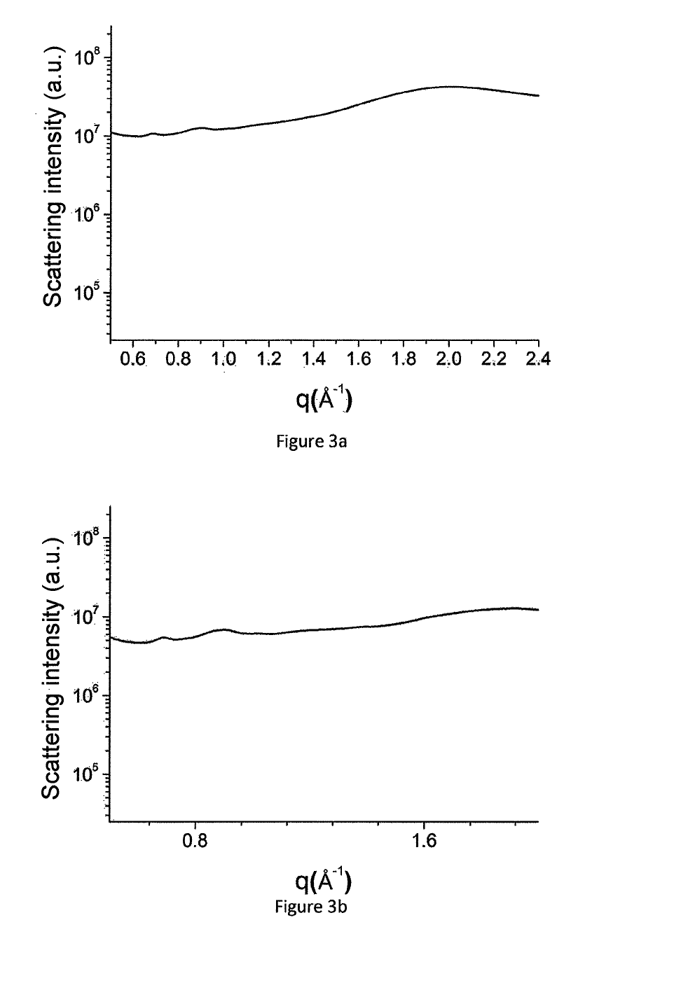

FIGS. 3a and 3b present the results of chitosan solutions which are not within the scope of the present invention, with a mixture of low DA and high DA, which are used as comparative products with the products according to the present invention.

FIG. 3a presents the intensity diffracted by a mixture of chitosan solutions of DA 15% and 40%, with respective concentrations of C=0.5 w % and 2 w %, after 72 hours in PBS.

FIG. 3b presents the intensity diffracted by a mixture of chitosan solutions of DA 15% and 55%, with respective concentrations of C=0.5 w % and 2 w %, after 72 hours in PBS.

A crystallinity peak representative of the line (200) of hydrated chitosan (see for example Osorio-Madrazo et al., Biomacromolecules 2010, 11, 1376-1386) around 1.40 Å⁻¹ is observed after 24 hours in PBS for a solution according to the present invention of low DA (2%, 3.5% and 15%), at a concentration of 3% by weight (see FIGS. 1a, 1b and 1c).

On the other hand, for the highest DAs (40% and 55%), which were tested on a purely comparative basis with the invention, no crystallinity is observed at 24 hours nor even at 72 hours; at longer times because the samples are solubilized in PBS and do not crystallize (see FIGS. 2a and 2b).

The solutions of low DA according to the invention, due to their greater ability to be crystallized, can thus be distinguished by a specific diffraction behavior, in spite of the very low polymer content of the solutions which are composed of more than 97% water.

After some time in PBS, the solutions of low DA thus become semicrystalline systems. The solutions of low DA according to the invention thus will have a longer filling effect due to the presence of this crystallinity, while the products of high DA will tend to be solubilized and degraded more quickly in tissues.

Solutions composed of chitosan of mixed DAs as described in the patent applications of the prior art WO 2008/072230 and WO 2009/150651 (see FIGS. 3a and 3b which show the results concerning mixed DA systems of 15% and 40%, and 15% and 55%, respectively) give results similar to solutions containing only chitosan of DA of 40% or 55% (see FIGS. 2a and 2b). These solutions do not crystallize under physiological conditions and are completely solubilized in PBS after 4 days.

### Example 2: In Vivo Study: Evaluation of Performance and Local Tolerance of Injectable Chitosan Solutions, Implanted Intradermally in Rabbits

The objective of the present study is the evaluation of the macroscopic local tolerance (by the evaluation of erythema, edema, necrosis and ulceration) and the performance (according to hardness and diameter criteria) of 6 test formulations, in comparison with 3 reference products, after intradermal implantation in rabbits.

Elements Tested:

The following compositions were tested: aqueous chitosan solutions, having a concentration of 3% by weight in mixture with 9‰ NaCl+0.3% lidocaine, sterilized by autoclave at 121° C. for 15 minutes.

The various DAs are obtained by reacetylation of a squid chitosan (Mahtani Chitosan Veraval, India) of DA 3.5%, Mw approximately 400,000 g/mol, purified by filtration of a chitosan acetate solution at a concentration of 0.5% by weight of polymer through a 0.45 μm filter. The solution is then lyophilized.

In a reactor, and with mechanical stirring (50 rpm), the chitosan lyophilizate is dissolved in deionized water using the stoichiometric amount of acetic acid necessary to the protonation of NH₂ sites. Various concentrations (0.5 w % to 3 w %) were studied.

The pH of each solution was controlled, and is in all cases between 5 and 5.5 (as a function of the chitosan concentration).

Test 1: chitosan C=3 w %, DA=5%

Test 2: chitosan C=3 w %, DA=15%

Test 3: chitosan C=3 w %, DA=40%

Test 4: chitosan C=3 w %, DA=55%

Test 5: chitosan C=3 w %, DA=5%+40%

Test 6: chitosan C=3 w %, DA=15%+55%

Reference Elements:
Ref. 1: Restylane® Perlane Lidocaine (29G needle), identified as: ref. 1—Restylane
Ref. 2: Ultra Juvederm® 4 (27G needle), identified as: ref. 2—Juvederm
Ref. 3: New Fill®/Sculptra (26G needle), identified as: ref. 3—NewFill.

Test System:
Species: rabbit
Strain: New Zealand white
Source: Charles River Laboratories
Health status: IOPS (SPF)
Number of animals: 8+1 reserve
Sex: female
Age on arrival: 18 weeks

Implantations:

Preimplant Procedures:

At Day 0, the animals were weighed, examined and then anaesthetized, according to the following protocol:

Ketamine (Ketamine 1000®—VIRBAC) 30 mg/kg (0.3 ml/kg)

+Medetomidine (Domitor®—Janssen Animal Health) 0.1 mg/kg (0.1 ml/kg)

Intramuscular (IM) injection in one thigh.

The dorsal zone was cropped with care (cropped again as needed thereafter for observations.)

Implantation Procedure:

Six injections were given per animal, on the dorsal zone. Care was taken not to inject too closely to the area of the nape of the neck and shoulders, so that the handling of the animal did not damage the sites.

Each site was marked by a tattoo, and then injected with 200 μl of product.

Follow-Up of Animals After Implantation:

Daily Observations

Observations were made each day by the personnel responsible for daily care (feeding, watering, cleaning, etc.).

They included a weighing of the animal, a complete physical examination, and a quick behavioral observation during handling.

Thorough Clinical Exams

Thorough clinical exams were carried out by the veterinarian, the Study Director or his deputy, generally when a consequent anomaly was noted during a daily observation or a basic clinical exam.

They included weighing, as well as the measuring of respiratory and cardiac rates, and the taking of rectal temperature.

The lymphatic, circulatory, respiratory, digestive, musculoskeletal and nervous systems, as well as the skin and the mucous membranes, were examined.

Macroscopic Observations

The observations took place at the following times:

Day 0 (post-implantation), T+24 hours, T+48 hours, Day 4.
The animal was photographed from above, while ensuring that the tattoo and all of the sites were visible.
The implantation sites were evaluated visually or manually by means of a scoring grid.
The diameter of the sites was measured using a caliper.

The parameters evaluated were: the formation of edema and erythema, the phenomena of ulceration and necrosis localized at the implantation sites, as well as the hardness and diameter of the sites.

Scale for Observations of Edema/Erythema/Ulcer/Necrosis:

(0) absent/(1) mild/(2) moderate/(3) marked/(4) severe

Euthanasia and Sampling:

At Day 2, five of the eight animals were anaesthetized and then given an intracardiac injection of sodium pentobarbital (Dolethal®—VETOQUINOL).

The implantation sites were removed and placed in labeled histology cassettes.

The samples from rabbits 1 to 3 were preserved in formol before treatment for histological study.

The samples from rabbits 4 and 5 were preserved in pure sterile water. A few hours later, the implant was extracted from the tissues for analysis under a synchrotron beam (WAXS technique, ESRF Grenoble, D2AM beamline). An explant fragment was placed in a capillary tube filled with water and then observed by synchrotron x-ray diffraction, using a 16 keV (λ=0.7749 Å) monochromatic beam.

At Day 4, the three remaining animals were anaesthetized and then given an intracardiac injection of sodium pentobarbital (Dolethal®—VETOQUINOL).

The implantation sites were removed and placed in labeled histology cassettes. These samples were preserved in formol before treatment for histological study.

Results:

Clinical Observations:

In all cases, during the first 4 days, edema and erythema increase as DA increases. The solutions with DA of 5% and 15% in certain animals induce edema and erythema scores of 0, while the solutions with DA≧40% result in edema and erythema scores of 3 or 4. The solutions with DA of 5% and 15% according to the present invention are the only ones that did not induce systematic necrosis at the implant sites.

It is important to note that the implant composed of chitosan with a DA of 55% is no longer palpable after 4 days, and that after 2 days it was not possible to remove it from the tissues for analysis by X-ray diffraction.

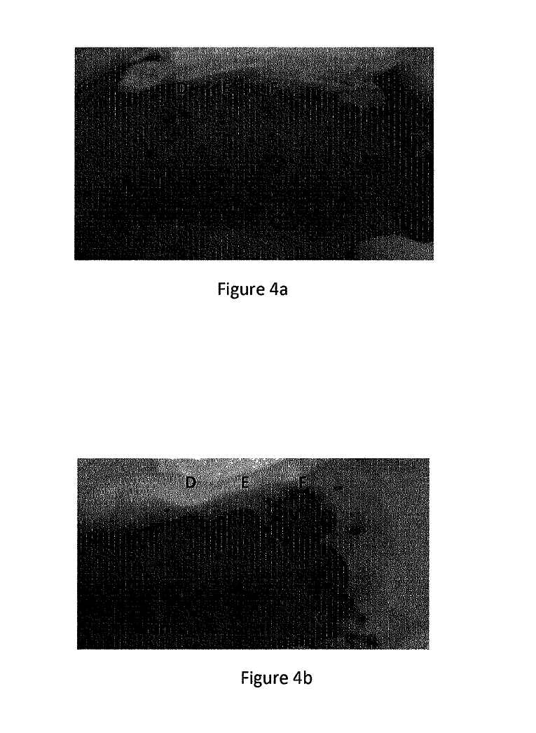

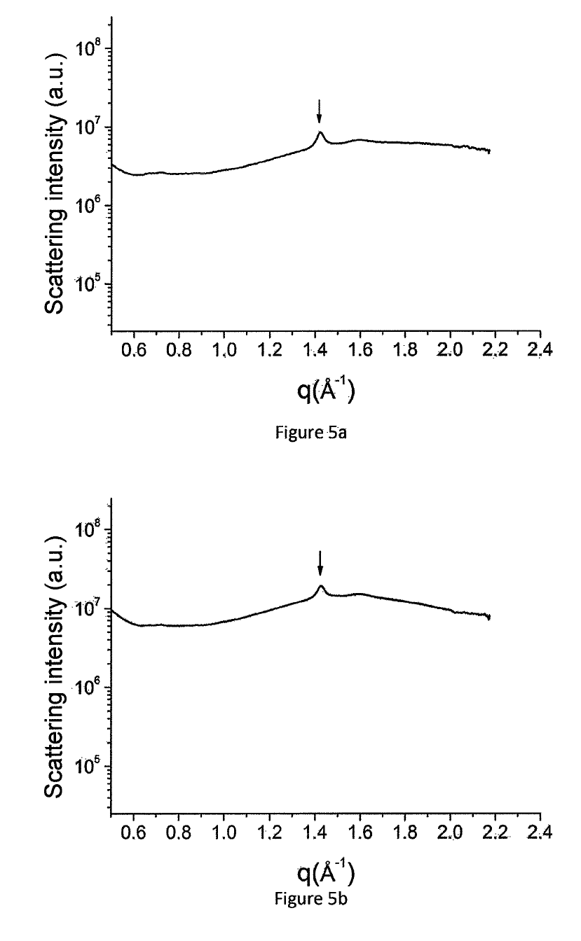

The example of two rabbits (R4 and R5) having received the “test” formulations is presented in FIGS. 4a and 4b.

FIGS. 4a and 4b show photographs of the injection sites of rabbits 4 and 5, respectively, 24 hours after implantation:

A: Test 1: chitosan C=3 w %, DA=5%

B: Test 2: chitosan C=3 w %, DA=15%

C: Test 3: chitosan C=3 w %, DA=40%

D: Test 4: chitosan C=3 w %, DA=55%

E: Test 5: chitosan C=3 w %, DA=5% +40%

F: Test 6: chitosan C=3 w %, DA=15%+55%

The solutions prepared according to the present invention induce a limited inflammatory response in comparison with the solutions containing chitosans of high DA or mixtures of chitosans containing in particular a high DA chitosan as described in patent applications WO 2008/072230 and WO 2009/150651. Moreover, as suggested by the complete disappearance of the implant with a degree of acetylation equal to 55% after only 4 days, the use of a high DA chitosan does not confer a satisfactory bioresorption time for the applications concerned.

Study of Explants by Synchrotron Beam

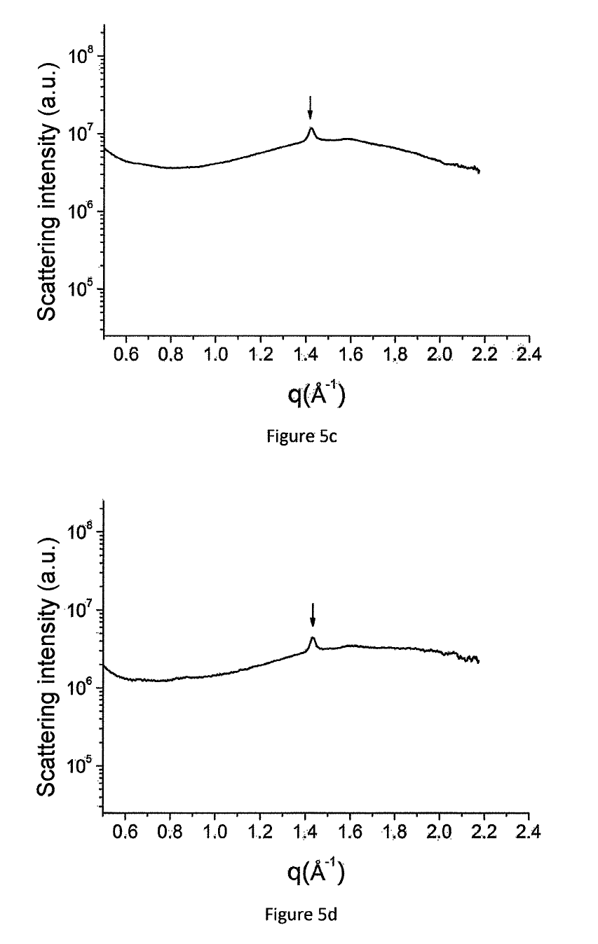

FIGS. 5 to 7 represent the results of the study by synchrotron x-ray diffraction beam of chitosan explants 24 hours after intradermal implantation of the solutions.

FIGS. 5a and 5b represent the results of a chitosan solution according to the invention (site A, test 1), with a low DA equal to 5%, after implantation of the solution in Rabbit 4 and Rabbit 5, respectively.

FIGS. 5c and 5d represent the results of a chitosan solution according to the invention (site B, test 2), with a low DA equal to 15%, after implantation of the solution in Rabbit 4 and Rabbit 5, respectively.

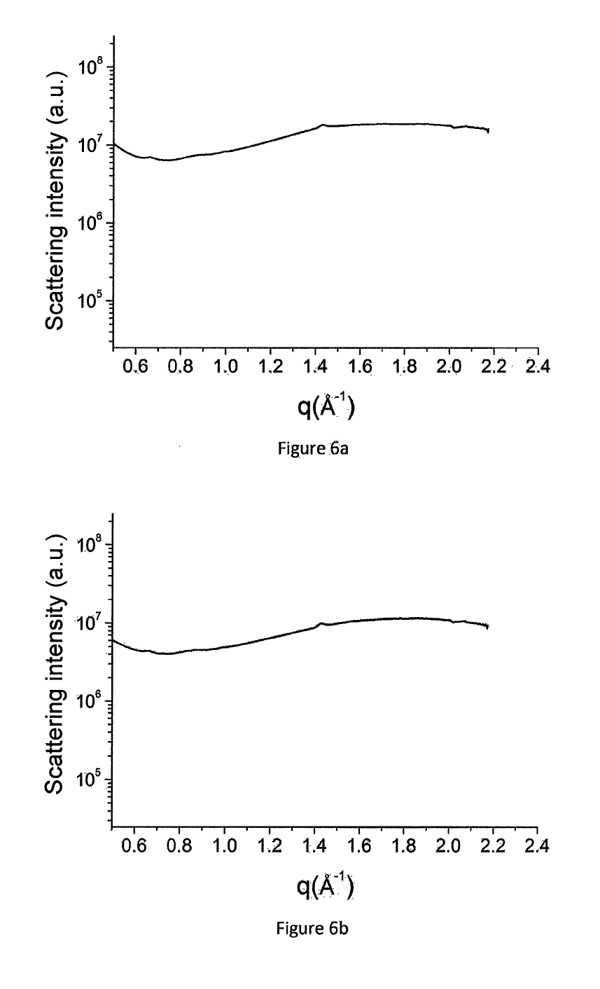

FIGS. 6a and 6b represent the results of a comparative chitosan solution (site C, test 3), with a high DA equal to 40%, after implantation of the solution in Rabbit 4 and Rabbit 5, respectively.

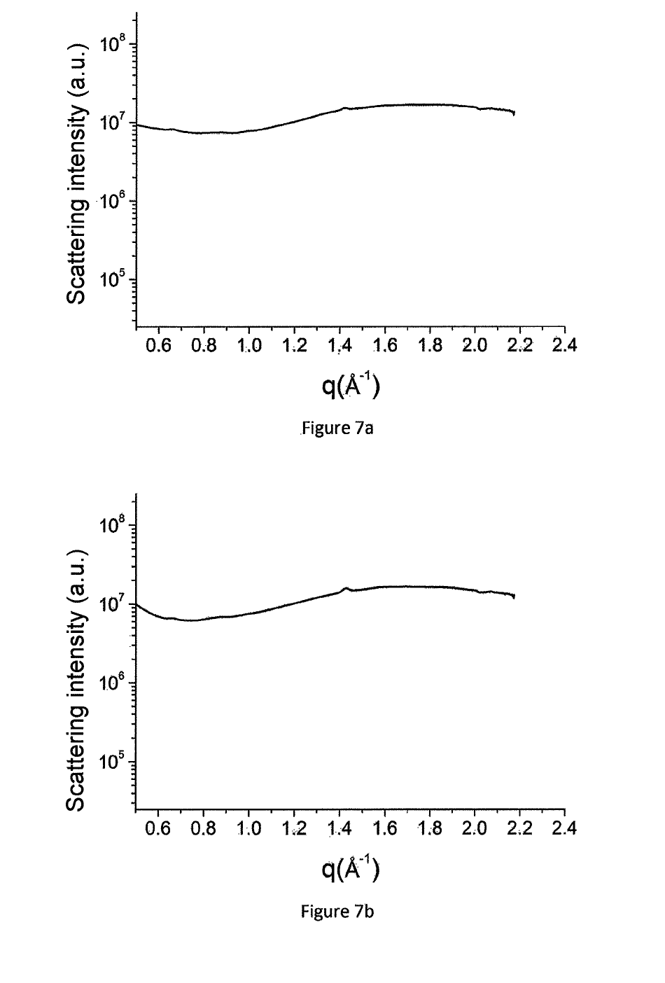

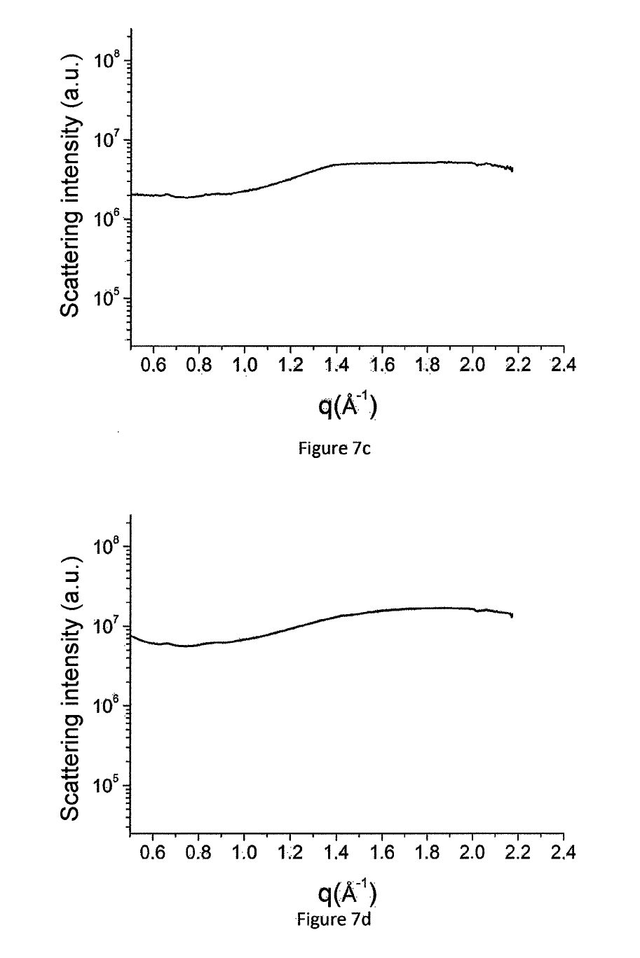

FIGS. 7a and 7b represent the results of a comparative chitosan solution (site E, test 5), with a mixture of low DA and high DA: 5%+40%, after implantation of the solution in Rabbit 4 and Rabbit 5, respectively.

FIGS. 7c and 7d represent the results of a comparative chitosan solution (site F, test 6), with a mixture of low DA and high DA: 15%+55%, after implantation of the solution in Rabbit 4 and Rabbit 5, respectively.

The intensity diffracted by the explants is given as a function of the scattering vector q=(4π sinθ)λ, where 2θ is the angle of diffraction (between the incident beam and the diffracted beam) and after subtraction of the intensity diffracted by the capillary tube filled with water alone, so as to best subtract the contribution of the water and the container to the diffraction.

In addition to the amorphous halo residue due to water, a well-defined crystallinity peak representative of the 200 line of hydrated chitosan around 1.40 Å⁻¹ for the explants of low DA (5% and 15%) is observed, whereas in the other cases it is weakly perceptible (DA 40%, DA mixture 5%+40%) or completely absent (DA 15%+55%). The low DA solutions, due to their greater ability to be crystallized, can thus be distinguished by a specific diffraction behavior, in spite of the very low polymer content of the solutions which are composed of more than 97% water.

After injection in the dermis, these low DA solutions thus become semicrystalline systems. A longer filling effect can thus be expected by virtue of the presence of this crystallinity, whereas the high DA products will tend to be solubilized and degraded more quickly in tissues.

The solutions composed of chitosan of mixed DAs as described in the patent applications of the prior art WO 2008/072230 and WO 2009/150651 give results similar to the solutions containing only chitosan of DA of 40% or 55%: these solutions do not crystallize, or crystallize very little, in tissues and, for the DA of 55%, they are no longer observable macroscopically within four days.

The crystallinity developed in situ indeed makes it possible to extend the bioresorption time, and is thus of great advantage for the applications concerned.

### Example 3: In Vivo Study: Evaluation of the Performance and Local Tolerance of Injectable Chitosan Solutions, Implanted Subcutaneously in Rats

The objective of the present study is the evaluation of macroscopic local tolerance (by the evaluation of erythema, edema, necrosis and ulceration) and of performance (according to hardness criteria) of 2 test formulations, in comparison with 1 reference product, after subcutaneous injection in rats.

Elements Tested:

The following compositions were tested: aqueous chitosan solutions, having a concentration of 3% by weight in mixture with 9‰ NaCl+0.3% lidocaine, sterilized by autoclave at 121° C. for 15 minutes.

The chitosan used is a squid chitosan (Mahtani Chitosan Veraval, India) of DA 2%, Mw (molar weight) approximately 400,000 g/mol, purified by filtration of a chitosan acetate solution at a concentration of 0.5% by weight of polymer through a 0.45 μm filter. The solution is then lyophilized.

In a reactor, and with mechanical stirring (50 rpm), the chitosan lyophilizate is dissolved in water for injection using the stoichiometric amount of acetic acid necessary to the protonation of NH₂ sites. The concentration 3 w % was studied.

The pH of each solution was controlled, and is in all cases between 5 and 6.2.

Test 1: chitosan C=3 w %, DA=2%, pH=5.

Test 2: chitosan C=3 w %, DA=2%, pH=6.

Reference Elements:
Ref 1: Restylane® Perlane Lidocaine (29G needle)

Test System:
Species: rat
Strain: Sprague Dawley
Number of animals: 6
Sex: male
Age on arrival: between 7 and 8 weeks
Weight on arrival: between 200 and 220 grams

Implantations:

Preimplant Procedures:

At Day 0, the animals were weighed, examined and then anaesthetized, according to the following protocol:
Intraperitoneal injection (1 ml/100 g of weight of the animal) of a sodium pentobarbital dilution (CEVA ANIMAL HEALTH—100 ml at 54.7 mg/ml) in a ratio of 6 ml for 44 ml of physiological saline.
The dorsal zone was cropped with care (cropped again as needed).
No antibiotic treatment was given.

Implantation Procedure:

Four subcutaneous injections using sterile glass syringes with sterile needles were given per animal, on the dorsal zone.

Each implantation site was marked by a tattoo, and then injected with 100 μl of product.

The injection sites were randomized with the criterion that each animal received at least one injection per formulation (test 1, test 2 and Ref. 1).

Follow-Up of Animals After Implantation:

Daily Observations

Observations were made each day by the personnel responsible for daily care (feeding, watering, cleaning, etc.).

They included a weighing of the animal, a complete physical examination, and a quick behavioral observation during handling.

Macroscopic Observations

The observations took place at the following times:

Day 0 (post-implantation), Day 2 (T+48 hours), Day 4 (T+96 hours).

The implantation sites were evaluated visually or manually by means of a scoring grid.

The parameters evaluated were: the formation of edema and erythema, the phenomena of ulceration and necrosis localized at the implantation sites, as well as hardness.

Scale for Observations of Edema/Erythema/Ulcer/Necrosis:

(0) absent/(1) mild/(2) moderate/(3) marked/(4) severe

Euthanasia and Sampling:

At Day 4, the animals were anaesthetized and then given a sodium pentobarbital injection (2 ml undiluted, IP). The implantation sites were removed so as to include the lesion and a contiguous uninjured zone, each sample comprising all the layers of the skin to the muscle. The samples were fixed in 4% aqueous formaldehyde solution for 48 hours.

Results:

Clinical Observations:

All the animals appeared and behaved normally during the entire observation period and their weight remained stable.

The visual and manual evaluation of the implant sites did not reveal any difference related to the formulations tested.

No erythemas induced by the solutions according to the present invention injected subcutaneously were observed.

The volumes observed are not related to an irritating effect of the solutions tested but are mechanical in origin (implant not reabsorbed).

In the experimental conditions adopted, the 2 solution formulations according to the present invention were well tolerated locally.

The evaluation of the macroscopic local tolerance and performance of test formulations 1 and 2 was comparable to that of the reference formulation Ref. 1.

No significant adverse reaction such as significant necrosis and ulceration of the skin was observed. The solutions prepared according to the present invention thus induce a limited inflammatory response.

Histological Examination of the Implants

The samples were fixed at least 24 hours before being dried.

One section (thickness 3 to 5 μm) was made per block. The slides were stained with hematoxylin-eosin.

Twenty-four virtual slides were analyzed.

The histological appearance of the implants is very different between the reference implant (Restylane/Perlane/Lidocaine) and the test implants (Test 1 and Test 2). Whereas the reference implant was homogeneous, the test implants had an appearance that was either micro-globular (variable diameter, generally between 5 and 15 μm—Test 1), or of the coagulum type (Test 2).

The reaction of the host to formulations Test 1 and Test 2 was generally limited to the hypodermis under the platysma muscle, consistent with fibroplasia/granulation tissue and mild-to-moderate inflammation surrounding the implant. This reaction consisted of granulation tissue rich in collagen fibers in the process of maturation and infiltration by mononucleated cells mostly consisting of monocytes/histiocytes and lymphocytes, with occasionally plasmocytes, but mostly without granulocytes.

Based on these criteria, a classification of weak reaction of the host (score 1) was observed for the reference item, an intermediate reaction (score 2) for formulations Test 1 and Test 2, with however a more marked host reaction in the case of item Test 1.

The solutions composed of chitosan according to the invention give results similar to the reference solution in terms of tolerance. On the other hand, just as in Example 2, after subcutaneous injection, these low DA solutions become semicrystalline systems. A longer filling effect by virtue of the presence of this crystallinity can thus be expected.

1. A homogeneous aqueous solution of injectable chitosan containing a chitosan having a degree of acetylation lower than 20%, advantageously lower than 10%, said solution containing between 0.1 and 3.5%, advantageously between 1 and 2.5%, by weight of chitosan, said solution having a pH lower than 6.2, advantageously between 5 and 6.2, said solution not containing a chitosan having a degree of acetylation higher than 20%, and said aqueous solution being capable of forming crystalline particles of chitosan after injection.

2. The aqueous solution according to claim 1, characterized in that the chitosan has a mean molecular weight between 100,000 and 1,000,000 g/mol, typically between 250,000 and 1,000,000 g/mol.

3. The aqueous solution according to any one of the preceding claims, characterized in that it can be prepared by the following steps:
dissolution of the chitosan in water by the addition of an acid such as a weak acid, said weak acid being advantageously selected from the group comprised of acetic acid, glycolic acid, lactic acid, glutamic acid, and mixtures thereof,
and, optionally, readjustment of the pH in order to obtain an aqueous solution having a pH between 5 and 6.2.

4. The aqueous solution according to claim 3, characterized in that, during the dissolution step, the acid is added in an amount strictly necessary to dissolve the chitosan.

5. A composition comprising an aqueous solution according to any one of the preceding claims, and optionally an acceptable compound or excipient, such as a compound or excipient to promote the crystallinity of the solution after injection, for example a short-chain chitosan of degree of acetylation lower than 20% and mean molecular weight lower than 20,000 g/mol, or a chito-oligosaccharide of degree of polymerization between 3 and 30.

6. The composition according to claim 5, further comprising at least an active compound such as an analgesic compound, local anesthetic, such as lidocaine, mepivacaine, bupivacaine or ropivacaine, an angiogenic compound, a vaccine, or an active compound of the growth factor or bioactive oligosaccharide type.

7. The composition according to claim 5 or 6, characterized in that it is formulated to be administered by intradermal or subcutaneous injection.

8. The composition according to any one of claims 5 to 7, for use as a dermatological or cosmetic composition, or as a medical device, advantageously as a bioresorbable implant.

9. The composition according to claim 8, for use in the repair or reconstruction of tissues of the skin of the face or body.

10. The composition according to claim 8 or 9, for use in the filling of facial cavities such as lines or wrinkles, for creating or increasing the volume of the human face or body, or for the cicatrization of the skin.

11. The composition according to claim 8, for use in surgery, in cosmetic medicine or surgery, in urology, rheumatology, ophthalmology, odontology, or in angiology.

12. The composition according to claim 8, for use as a vector for active ingredients, for example as a carrier of vaccines or hormones.

13. A cosmetic use of an aqueous solution according to any one of claims 1 to 4 or of a composition according to claim 5 to treat or prevent skin aging.

14. Use of a homogeneous aqueous solution of injectable chitosan containing a chitosan having a degree of acetylation lower than 20%, advantageously lower than 10%, said solution containing between 0.1 and 3.5%, advantageously between 1 and 2.5%, by weight of chitosan, said solution having a pH lower than 6.2, advantageously between 5 and 6.2, to form crystalline particles of chitosan after injection.

15. Use according to claim 14, characterized in that the chitosan has a mean molecular weight between 100,000 and 1,000,000 g/mol, typically between 250,000 and 1,000,000 g/mol.
